(19) United States
(12) Patent Application Publication
(10) Pub. No.: US 2014/0363530 A1
(43) Pub. Date: Dec. 11, 2014

(54) COMPOSITION COMPRISING THE EXTRACT OF PINE TREE LEAF OR THE COMPOUNDS ISOLATED THEREFROM FOR THE PREVENTION AND TREATMENT OF CANCER DISEASE BY INHIBITING HPV VIRUS AND THE USES THEREBY

(71) Applicants: Chiung MOON, (US); GUEULRI, Ansan-si, Gyeonggi-do (KR)

(72) Inventors: Chiung Moon, Ansan-si (KR); Jong Hwan Kwak, Suwon-si (KR); Young Bong Kim, Goyang-si (KR); Yukyoung Jeon, Seoul (KR); Hee-Jung Lee, Seoul (KR)

(73) Assignees: GUEULRI, Ansan-si, Gyeonggi-do (KR); Chiung MOON, Ansan-si, Gyeonggi-do (KR)

(21) Appl. No.: 14/366,093

(22) PCT Filed: May 29, 2013
(86) PCT No.: PCT/KR2013/004698

§ 371 Date: Jun. 17, 2014

(30) Foreign Application Priority Data
May 31, 2012 (KR) 10-2012-0058332
May 31, 2012 (KR) 10-2012-0058333
May 31, 2012 (KR) 10-2012-0058334
May 31, 2012 (KR) 10-2012-0058335
May 10, 2013 (KR) 10-2013-0053204
May 10, 2013 (KR) 10-2013-0053205

(51) Int. Cl. C07C 62/06; C07C 62/32; A23L 1/30; A61K 36/15

(52) U.S. Cl. CPC C07C 62/06; A61K 36/15; C07C 62/32; A23L 1/3002; A23V 2002/00
USPC 424/770; 562/499; 514/557

(57) ABSTRACT

A composition contains an extract of pine leaf extract or a compound isolated therefrom. It showed potent inhibitory effect on human papillomavirus (HPV) as well as anti-cancer effect on various cancer diseases through various in vitro test and in vivo tests, and therefore, it can be used as the effective and safe therapeutics or health food for treating and preventing cancer disease.

### TECHNICAL FIELD

The present invention relates to a composition comprising the extract of pine tree leaf or the compounds isolated therefrom for the prevention and treatment of cancer disease by inhibiting HPV virus and the uses thereby.

### BACKGROUND ART

Cancer has been regarded as serious clinical problem and exerts an important social and economical effect on the human health care system. As a carcinogenic substance to cause cancer disease, smoking, ultraviolet ray, chemical substance, food and other environmental factors have been reported till now however the etiology of cancer is diverse, which results in difficulty in development of therapeutics as well as unequal potency of therapeutics according to the occurring region of cancer. Since presently used cancer drugs show considerable adverse effect and could not treat cancer selectively, there have been still needed to develop potent anticancer drug with little toxicity to treat and prevent cancer disease till now.

Particularly, more than 90% cervical cancer diseases are mostly caused by malignant tumor virus and are reported to show the highest occurrence rate and death rate among Korean women. Recently, human papillomaviruses (HPV) infection also has been reported to play the most important roles in the mechanism of oncogenesis.

Uterine cancer has been reported to show the highest occurrence rate in the various cancers of women located in developing countries and approximate five hundred thousand patients has been found every year (Vanchieri, C., IARC Publishes Data on Worldwide Cancer Cases, Journal of the National Cancer Institute, 85(13), pp 1028-1029, 1993: Munoz, N. and Bosch, F. X., Epidemiology of cervical cancer. In: Human papillomaviruses and Cervical Cancer, eds. Munoz, N., Bosch, F. X. and Jensen, O. M. IARC Scientific Publications, Lyon, pp. 9-40, 1989).

Papillomavirus has been reported to infect on the epithelial cell of various animal tissue and give rise to benign tumors such as wart occurring at hand, foot, skin, etc. 70 kinds of genotype have been found in human papillomavirus and it has specificity to each infected tissue, resulting in various disease (Broker et al., Papillomaviruses: Retrospectives and Prospectives, Cancer cells 4/DNA tumor viruses, Cold Spraing Harbor Laboratory, USA, pp 17-36, 1989).

The various genotypes of papillomavirus has not been paid attention till now since they do not occur lethal disease although they inflict great infliction on patients due to the difficulty in efficient treatment. However the specific genotype of them, in particular, genotype 16 and 18 papillomaviruses have been found to be involved in the malignant tumors in various tissues for example, genitals of man and woman, oral cavity, skin etc and to act as a main etiological factor to cause detrimental cervical cancers as well as benign tumors occurring in the genitals of man and woman called as “Condyloma acuminata” in case of genotypes 6b and 11b. Through epidemiological survey, cervical cancers are reported to be occurred by the transmitted factor of sexual intercourse (Durst, M. et al., Papillomavirus DNA from a cervical carcinoma and its prevalence in cancer biopsy samples from different geographical regions, Proc. Natl. Acad. Sci., USA, 80. 3812-9-2 1019950024886, 3815 (1983)). It has been found that papillomavirus is closely correlated with the occurrence of cervical cancer, which had been demonstrated by several experiments, for example, about 85-100% precancerous lesion called as cervical intraepithelial neoplasm (CIN), is infected with papillomavirus (Hausen, H., Viruses in human cancers, Science, 254, 1173-1187 (1991)). Accordingly, there have been needed to the development for the mechanism of oncogenesis, as well as the diagnostic tool using thereby and the treating agent (Galloway, D. A. et al., Human papillomaviruses and carcinomas, Adv. Virus Res., 37, 125-171 (1990)).

There have been reported that the infection rate of human papillomaviruses type 16 in the patients suffering from cervical cancer showed 50-70% and type 16 showed 15-25% but in case of the patients suffering from metastatic cancer, type 16 showed 25% and type 18 showed more than 50% respectively (Lorincz, A. T. et al., Human Papillomavirus Infection of the Cervix: Relative Risk Associations of 15 Common Anogenital Types, Obsterics and Gynecology, 79, 328-337 (1992)).

Although various therapeutic methods such as chemotherapeutics, radiotherapeutics, surgical therapy, gene therapy and the like have been used now, the chemotherapeutics among them has been mostly wisely used. However it gives rise to lots of adverse response and does not provide complete treatment therefore new approach has been needed to treat cancer disease. Those approach can be classified into two ways, I. e., one way is to synthesize and develop new chemotherapeutic derivatives based on known chemotherapeutics showing significantly decreased adverse response with similar potency to the known chemotherapeutics through diverse synthetic methods and another way is a cancer chemoprevention to prevent the progress to malignant tumor by way of inhibiting from cancer occurrence or postponing or reversing cancer development.

Recently, there have been much interested in the development of chemo-preventive and chemo-therapeutic substance showing potent potency with low toxicity from natural resources such as vegetable, fruit, medicinal herb etc in Western countries (Kelloff et al., Annals New York Academy of Sciences, 889, pp1-13, 1999).

Pine leaf is widely distributed in South Korea and it has been used in treating various disease as a folk remedy in Korea, for example, insomnia treating agent, diuretic, analgesic, anti-inflammatory agent, anthelmintics, etc, (Kim, T. J., Korean Resources Plants. II. pp 194-195, 1996).

Recently, there have been reported that pine leaf contains various chemical ingredients (Kang, T. H., Jeong, S. T., et al., Journal of Ethnopharmacology, 71, pp 321-323, 2000; Jung, M. J., Chung, H. Y. et al., Archives of pharmacal research, 26(6), pp 458-462), for example, quercetin 3-O-galactoside, quercetin 3-O-rhamnoside, 3,4,7-trihydroxyflavonea-spinastery glucoside, triterpenoid, saponin, and lignan etc in leaf (Kaneta M., Hikichi H. et al., Agricultural and Biological Chemistry, 44, pp 1407, 1980; Woo W. S., Kang S. S. et al., Journal of Natural Products, 49, pp 547-549, 1984); flavonol glycosides such as quercitrin, isoquercitrin etc in the pine flower. There have been also reported on the pharmacological activities of pine leaf extract, for example, oxitocic activity of saponin fraction isolated from pine leaf extract (Woo W. S., Lee E. B. et al., Kor. J. Pharmacogn, 12 pp 153, 1981); anti-bacterial activity (Yang M. S., Ha Y. L. et al., Agricultural Chemistry and Biotechnology, 38, pp 584, 1995); diarrhea inhibitory activity (Yoo J. S., Jang J. S. et al., KOR. J. Pharmacogn, 26, pp 355, 1995), and hypnotic activity of flavonoid glycoside isolated from pine leaf extract etc.

About 80-90 species of pine tree beloned to Pinus genus have been distributed over the world and some terpene components isolated from several species, for example, Pinus palustris Miller (North America), P. pinaster Aiton (France), P. sylvestris L. (Europe), P. laricid Poiret (Australia), P. longifolia Rocvurgh (India), P. densiflora Sieb. et Zucc. (South Korea and Japan), P. thunberii Palatore (Japan) etc have been industrially used.

However, there has been not reported or disclosed on the therapeutic effect of the extract of pine leaf or the compounds isolated therefrom on the cancer disease, especially, cervical cancer by inhibiting HPV virus in any of above cited literatures, the disclosures of which are incorporated herein by reference.

Accordingly, the present inventors have confirmed that the extract of pine leaf extract or the compound isolated therefrom showed potent inhibitory effect on HPV virus as well as anti-cancer effect on various cancer diseases through various in vitro test and in vivo tests, for example, inhibitory activity of luciferase-containing HPV virus contagion (SEAP screening test; Experimental example 1); inhibitory effect on HPV16 PVs (Experimental example 2); inhibitory effect on various human tumor cell lines, such as human lung cancer cell line (A-549), human ovarian tumor cell line (SK-OV-3), human malignant melanoma cell line (SK-MEL-2), colonic adenocarcinoma cell line (HCT15), human cervical cancer cell line (MES-SA) and human resistant cervical cancer cell line (MES-SA/DX5) etc (Experimental example 3); in vivo inhibitory activity of HPV16 pseudo virus in mice (Experimental example 4), therefore, it can be used as the effective and safe therapeutics or health food for treating and preventing cancer disease.

### DISCLOSURE OF INVENTION

### Technical Problem

According to one aspect of the present invention, the present invention provides a composition comprising the extract of pine leaf or the compound isolated therefrom for the prevention and treatment of cancer disease, especially, cervical cancer disease caused by HPV virus.

According to another aspect of the present invention, the present invention provides a novel compound or the pharmacologically acceptable salt thereof having potent anti-cancer activity.

According to the other aspect of the present invention, the present invention provides a method of treating or preventing cancer disease, especially cervical cancer disease, in human or mammal, wherein the method comprises administering a therapeutically effective amount of the extract of pine leaf or the compound isolated therefrom, as an effective ingredient, together with a pharmaceutically acceptable carrier thereof.

According to the other aspect of the present invention, the present invention provides a use of the extract of pine leaf or the compound isolated therefrom for the preparation of therapeutic agent for the treatment and prevention of cancer disease, especially cervical cancer disease, in mammal or human.

### Solution to Problem

Accordingly, it is an object of the present invention to provide a pharmaceutical composition comprising the extract of pine leaf, the compound isolated therefrom selected from 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) or the pharmacologically acceptable salt thereof, for the prevention and treatment of cancer disease, especially, cervical cancer disease caused by HPV virus.

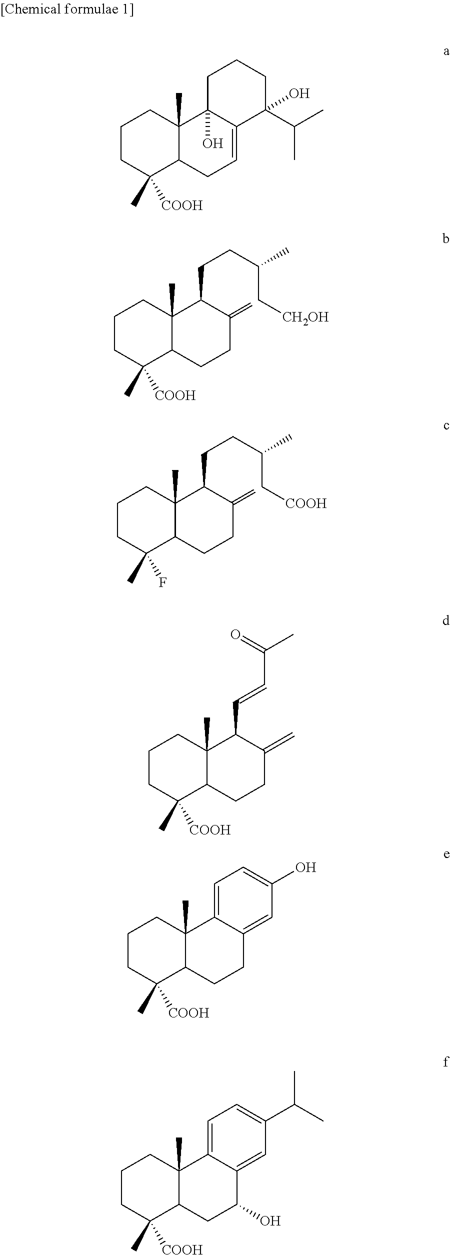

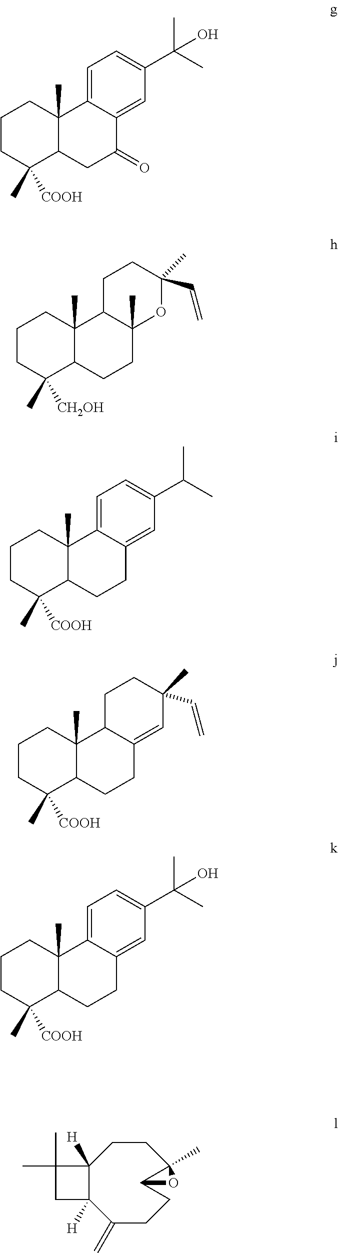

It is another object of the present invention to provide a novel 9,14-dihydroxytotara-7-ene-8-oic acid of chemical formulae (a) or the pharmacologically acceptable salt thereof having potent anti-cancer activity.

It is another object of the present invention to provide a novel (13S)-15-hydroxylabd-8(17)-en-18-oic acid of chemical formulae (b) or the pharmacologically acceptable salt thereof having potent anti-cancer activity.

It is the other object of the present invention to provide a health functional food composition comprising the extract of pine leaf, the compound selected from 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) or the pharmacologically acceptable salt thereof, isolated therefrom for the prevention and alleviation of cancer disease, especially, cervical cancer disease caused by HPV virus.

The term “treatment and prevention of cervical cancer disease caused by HPV” disclosed herein is performed by way of inhibiting HPV virus.

The term “pine tree” disclosed herein comprises Pinus densiflora Sieb. et Zucc, P. rigida, P. taeda, P. thunberii Palatore, P. koraiensis Sieb. et Zucc, Pinus palustris Miller, Pinus palustris Miller, P. pinaster Aiton, P. sylvestris L., P. laricid Poiret, and P. longifolia Rocvurgh, etc, preferably, Pinus densiflora Sieb. et Zucc, P. rigida, and P. taeda.

The term “extract” disclosed herein comprises crude extract, polar solvent soluble extract, non-polar solvent soluble extract and purified extract of pine tree leaf.

Specifically, the term “crude extract” disclosed herein comprises the extract prepared by extracting plant material with water, lower alcohols such as methanol, ethanol, or the mixtures thereof, preferably, water or 30-90% ethanol, more preferably, 50-80% ethanol soluble extract, more specifically, specifically, which can be prepared by adding 1 to 20-fold, preferably, approximately 1 to 7-fold volume of distilled water, C₁ to C₄ lower alcohols or the mixtures thereof, preferably the mixture of water and ethanol to dried pine tree leaf to perform to extraction method selected from hot water extraction, cold water extraction, reflux extraction, or ultra-sonication extraction, preferably, hot water extraction or cold water extraction at the temperature ranging from 10° C.˜150° C., preferably, 20° C.˜100° C., for the period ranging from 12 hours to 1 week, preferably, 24 hours to 72 hours to extract at 1st step; filtering the solution to afford the filtrate at 2nd step; concentrating the filtrate to afford the crude extract of the present invention.

Specifically, the term “non-polar solvent soluble extract” disclosed herein can be soluble in non-polar solvent, for example, hexane, methylene chloride, ethyl acetate or chloroform, preferably methylene chloride, specifically, which can be prepared by suspending the crude extract in 0.005 to 10-fold volume (v/w), preferably, 0.05 to 0.5-fold volume (v/w) of distilled water, and fractionating the suspension with the above-described non-polar solvent repeatedly to afford the non-polar solvent soluble extract of the present invention.

Specifically, the term “polar solvent soluble extract” disclosed herein can be soluble in polar solvent, for example, water, lower alcohol such as methanol, ethanol, preferably butanol and water, specifically, which can be prepared by fractionating the above-described crude extract with the above-described polar solvent with removing non-polar solvent soluble extract at 1st step; and collecting the polar solvent soluble extract at 2nd step to afford polar solvent soluble extract of the present invention.

Specifically, the term “purified extract” disclosed herein comprises (1) a purified extract eluted with water (designated as “S11-HPO” hereinafter), (2) a purified extract eluted with 30% ethanol (designated as “S11-HP30” hereinafter), (3) a purified extract eluted with 50% ethanol (designated as “S11-HP50” hereinafter), (4) a purified extract eluted with 70% ethanol (designated as “S11-HP70” hereinafter), (5) a purified extract eluted with 95% ethanol (designated as “S11-HP95” hereinafter), and (6) a purified extract eluted with acetone and methylene chloride (designated as “S11-HPAM” hereinafter) using by adsorbent resin and eluting solvent with decreasing the polarity of the solvent starting from water, ethanol, acetone to methylene chloride, serially; specifically, which can be prepared by adding about 1-30 fold weight (w/w), preferably, 5-15 fold weight (w/w), more preferably, 8-12 fold weight (w/w) distilled water to the crude extract of pine tree leaf at 1st step; performing adsorption purification method using by adsorbent resin such as SP207, HP20SS, Diaion HP 20, SP-850 resin, active carbon, or Amberlite XAD-2,4, preferably, Diaion HP 20, SP-850 resin, or Amberlite XAD-2,4 and eluting the column with the eluting solvent with decreasing the polarity of the solvent starting from water, ethanol, acetone to methylene chloride, serially, to afford (1) a purified extract eluted with water (designated as “S11-HPO” hereinafter), (2) a purified extract eluted with 30% ethanol (designated as “S11-HP30” hereinafter), (3) a purified extract eluted with 50% ethanol (designated as “S11-HP50” hereinafter), (4) a purified extract eluted with 70% ethanol (designated as “S11-HP70” hereinafter), (5) a purified extract eluted with 95% ethanol (designated as “S11-HP95” hereinafter), and (6) a purified extract eluted with acetone and methylene chloride (designated as “S11-HPAM” hereinafter) of the present invention.

Accordingly, it is the other object of the present invention to provide a method for preparing the extract of pine tree leaf and the composition comprising the same prepared by the above-described method.

Also, the above-described procedures may be modified or subjected to further step to fractionate or isolate more potent fractions or compounds by conventional procedure well-known in the art, for example, the procedure disclosed in the literature (Harborne J. B. Phytochemical methods: A guide to modern techniques of plant analysis, 3^{rd} Ed. pp 6-7, 1998).

The term “cancer disease” disclosed herein comprise various cancer disease, for example, cervical cancer, resistant cervical cancer, lung cancer, ovarian tumor, malignant melanoma, colonic cancer, colon cancer or rectal cancer, bone cancer, pancreatic cancer, skin cancer, cancer of the head and neck, cutaneous or intraocular melanoma, uterine cancer, ovarian cancer, rectal cancer or cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (eg., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphonas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas), preferably solid cancer such as cervical cancer, resistant cervical cancer, lung cancer, ovarian tumor, malignant melanoma, colonic cancer or cancer of the head and neck, more preferably, cervical cancer, resistant cervical cancer, lung cancer, ovarian tumor, malignant melanoma, or colonic cancer.

The inventive compounds of the present invention may be chemically synthesized by the methods well-known in the art or be isolated from the extract of pine tree leaf which will be explained as follows, which are merely exemplary and in no way limit the invention.

For example, the above-described non-polar solvent soluble extract is purified with silica gel column chromatography method using by various solvent systems, i.e., (1) hexane: methylene chloride (1:1), (2) methylene chloride, (3) hexane: methylene chloride:methanol (10:10:0.5, 10:10:2) and (4) methylene chloride:methanol (1:1); the collected fractions are further purified with silica gel column chromatography method using by various solvent systems, i.e., (1) hexane:ethyl acetate (20:1, 10:1, 5:1, 3:1), (2) methylene chloride:methanol (3:1), (3) 100% methanol; and the selected fractions are further purified with silica gel column chromatography method using by various solvent systems, i.e., (1) hexane:ethyl acetate (7:11:1), (2) hexane:ethyl acetate:methanol (10:10:5), (3) methylene chloride:methanol (1:1), (3) 100% methanol; and the resulting sub-fractions are further purified with silica gel column chromatography method using by solvent system, i.e., hexane:ethyl acetate (10:1), and with LiChroprep RP-18 (40-63 μm, Merck, U.S.A.) chromatography (eluting with 80% methanol) or Sephadex LH-20 (eluting with methylene chloride:methanol=4:6) chromatography to afford 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) of the present invention.

The inventive compounds of the present invention can be transformed into their pharmaceutically acceptable salt and solvates by the conventional method well known in the art. For the salts, acid-addition salt thereof formed by a pharmaceutically acceptable free acid thereof is useful and can be prepared by the conventional method. For example, after dissolving the compound in the excess amount of acid solution, the salts are precipitated by the water-miscible organic solvent such as methanol, ethanol, acetone or acetonitrile to prepare acid addition salt thereof and further the mixture of equivalent amount of compound and diluted acid with water or alcohol such as glycol monomethylether, can be heated and subsequently dried by evaporation or filtrated under reduced pressure to obtain dried salt form thereof.

As a free acid of above-described method, organic acid or inorganic acid can be used. For example, organic acid such as methansulfonic acid, p-toluensulfonic acid, acetic acid, trifluoroacetic acid, citric acid, maleic acid, succinic acid, oxalic acid, benzoic acid, lactic acid, glycolic acid, gluconic acid, galacturonic acid, glutamic acid, glutaric acid, glucuronic acid, aspartic acid, ascorbic acid, carbonylic acid, vanillic acid, hydroiodic acid and the like, and inorganic acid such as hydrochloric acid, phosphoric acid, sulfuric acid, nitric acid, tartaric acid and the like can be used herein.

Further, the pharmaceutically acceptable metal salt form of inventive compounds may be prepared by using base. The alkali metal or alkali-earth metal salt thereof can be prepared by the conventional method, for example, after dissolving the compound in the excess amount of alkali metal hydroxide or alkali-earth metal hydroxide solution, the insoluble salts are filtered and remaining filtrate is subjected to evaporation and drying to obtain the metal salt thereof. As a metal salt of the present invention, sodium, potassium or calcium salt are pharmaceutically suitable and the corresponding silver salt can be prepared by reacting alkali metal salt or alkali-earth metal salt with suitable silver salt such as silver nitrate.

The pharmaceutically acceptable salt of the present invention comprise all the acidic or basic salt which may be present at the compounds, if it does not indicated specifically herein. For example, the pharmaceutically acceptable salt of the present invention comprise the salt of hydroxyl group such as the sodium, calcium and potassium salt thereof; the salt of amino group such as the hydrogenbromide salt, sulfuric acid salt, hydrogen sulfuric acid salt, phosphate salt, hydrogen phosphate salt, dihydrophosphate salt, acetate salt, succinate salt, citrate salt, tartarate salt, lactate salt, mandelate salt, methanesulfonate(mesylate) salt and p-toluenesulfonate (tosylate) salt etc, which can be prepared by the conventional method well known in the art.

It is confirmed that the extract of pine leaf extract or the compound isolated therefrom showed potent inhibitory effect on human papillomavirus (HPV) as well as anti-cancer effect on various cancer diseases through various in vitro test and in vivo tests, for example, inhibitory activity of luciferase-containing HPV virus contagion (SEAP screening test; Experimental example 1); inhibitory effect on HPV16 PVs (Experimental example 2); inhibitory effect on various human tumor cell lines, such as human lung cancer cell line (A-549), human ovarian tumor cell line (SK-OV-3), human malignant melanoma cell line (SK-MEL-2), colonic adenocarcinoma cell line (HCT15), human cervical cancer cell line (MES-SA) and human resistant cervical cancer cell line (MES-SA/DX5) etc (Experimental example 3); in vivo inhibitory activity of HPV 16 pseudo virus in mice (Experimental example 4), therefore, it can be used as the effective and safe therapeutics or health food for treating and preventing cancer disease.

Moreover, the pine tree leaf extract of the present invention has been used as a folk remedy therefore, the extract of pine leaf, and the compounds isolated therefrom can be safely used a medicament or food with potent pharmacological activity and little toxicity.

Accordingly, the present invention also provided a pharmaceutical composition comprising the extract of pine leaf, the compound selected from 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) or the pharmacologically acceptable salt thereof, isolated therefrom prepared by the above-described preparation method as an active ingredient and a pharmaceutically acceptable carrier thereof for treating and preventing cancer disease.

According to the other aspect of the present invention, the present invention provides a use of the extract of pine leaf, the compound selected from 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) or the pharmacologically acceptable salt thereof, isolated therefrom for the preparation of therapeutic agent for the treatment and prevention of cancer disease, especially cervical cancer disease, in mammal or human.

According to the other aspect of the present invention, the present invention also provides a method of treating or preventing cancer disease, especially cervical cancer disease, in human or mammal, wherein the method comprises administering a therapeutically effective amount of the extract of pine leaf, the compound selected from 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) or the pharmacologically acceptable salt thereof, isolated therefrom, as an effective ingredient, together with a pharmaceutically acceptable carrier thereof.

The inventive composition for treating and preventing purposed diseases may comprises the above-described compound as 0.02-50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 0.1 to 1000 mg/kg, preferably, 1 to 100 mg/kg by weight/day of the inventive extract of the present invention. The dose may be administered in single or divided into several times per day. In terms of composition, the amount of inventive extract should be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intra-cutaneous, intrathecal, epidural or intra-cerebroventricular injection.

The inventive extract or compound of the present invention also can be used as a main component or additive and aiding agent in the preparation of various functional health food and health care food.

Accordingly, it is the other object of the present invention to provide a health functional food comprising the extract of pine leaf, the compound selected from 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) or the pharmacologically acceptable salt thereof, isolated therefrom for the prevention or alleviation of cancer disease, especially cervical cancer disease.

The term “a functional health food” defined herein “the functional food having enhanced functionality such as physical functionality or physiological functionality by adding the compound of the present invention to conventional food to prevent or improve cerebrovascular system involved anxiety in human or mammal.

It is the other object of the present invention to provide a health care food comprising the extract of pine leaf, the compound selected from 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l) or the pharmacologically acceptable salt thereof, isolated therefrom, together with a sitologically acceptable additive for the prevention or alleviation of cancer disease, especially cervical cancer disease.

The term “a health care food” defined herein means the food containing the compound of the present invention showing no specific intended effect but general intended effect in a small amount of quantity as a form of additive or in a whole amount of quantity as a form of capsule, pill, tablet etc.

The term “a sitologically acceptable additive” defined herein “any substance the intended use which results or may reasonably be expected to result-directly or in-directly-in its becoming a component or otherwise affecting the characteristics of any food” for example, thickening agent, maturing agent, bleaching agent, sequestrant, humectant, anti-caking agent, clarifying agents, curing agent, emulsifier, stabilizer, thickener, bases and acid, foaming agents, nutrients, coloring agent, flavoring agent, sweetener, preservative agent, anti-oxidant, etc, which shall be explained in detail as follows.

If a substance is added to a food for a specific purpose in that food, it is referred to as a direct additive and indirect food additives are those that become part of the food in trace amounts due to its packaging, storage or other handling.

Above described health care foods can be contained in food, health beverage, dietary supplement etc, and may be used as a form of powder, granule, tablet, chewing tablet, capsule, beverage etc for preventing or improving of purposed disease.

Also, above described extract or compound can be added to food or beverage for prevention and improvement of purposed disorder. The amount of above described extract or compound in food or beverage as a functional health food or health care food may generally range from about 0.01 to 100 w/w % of total weight of food for functional health food composition. In particular, although the preferable amount of the compound of the present invention in the functional health food, health care food or special nutrient food may be varied in accordance to the intended purpose of each food, it is preferably used in general to use as an additive in the amount of the extract or compound of the present invention ranging from about 0.01 to 5% in food such as noodles and the like, from 40 to 100% in health care food on the ratio of 100% of the food composition.

Providing that the health beverage composition of present invention contains above described extract or compound as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudiosideA, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract or compound therein are various food, beverage, gum, vitamin complex, health improving food and the like.

Inventive extract or compound of the present invention has no toxicity and adverse effect therefore; they can be used with safe.

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### Advantageous Effects of Invention

The present invention comprising the extract of pine leaf extract or the compound isolated therefrom showed potent inhibitory effect on HPV virus as well as anti-cancer effect on various cancer diseases through various in vitro test and in vivo tests, for example, inhibitory activity of luciferase-containing HPV virus contagion (SEAP screening test; Experimental example 1); inhibitory effect on HPV16 PVs (Experimental example 2); inhibitory effect on various human tumor cell lines, such as human lung cancer cell line (A-549), human ovarian tumor cell line (SK-OV-3), human malignant melanoma cell line (SK-MEL-2), colonic adenocarcinoma cell line (HCT15), human cervical cancer cell line (MES-SA) and resistant human cervical cancer cell line (MES-SA/DX5) etc (Experimental example 3); in vivo inhibitory activity of HPV 16 pseudo virus in mice (Experimental example 4), therefore, it can be used as the effective and safe therapeutics or health food for treating and preventing cancer disease.

### BRIEF DESCRIPTION OF DRAWINGS

The above and other objects, features and other advantages of the present invention will more clearly understood from the following detailed description taken in con-junction with the accompanying drawings, in which;

FIG. 1 shows the inhibitory effect (in vitro) of test samples (extract and fractions) against HPV16 PVs;

FIG. 2,3 shows the inhibitory effect (in vitro) of test samples (selected fractions) against HPV16 PVs by pre-treatment time;

FIG. 4 shows the inhibitory effect (in vitro) of test samples (isolated compounds against HPV16 PVs;

FIG. 5 shows the protective effect (in vivo) of test samples (selected fractions) against HPV16 PVs challenge according to administration methods;

### BEST MODE FOR CARRYING OUT THE INVENTION

The following Reference Example, Examples and Experimental Examples are intended to further illustrate the present invention without limiting its scope.

### Example 1

### Preparation of Pine Tree Leaf Extract

1-1. Crude Extract of Pine Tree Leaf

2 kg of dried power of pine leaf purchased from Kyung Dong Herbal Market (Seoul, Korea), were added to 20 L of 95% ethanol and the solution was left alone for 3 days with stirring at 50° C. The residue was filtered and the extraction process was repeated two times. The filtrate was collected and concentrated to obtain 338.85 g of 95% ethanol soluble extract of pine tree leaf (designated as “S11-T”, hereinafter).

1-2. Preparation of Solvent Soluble Extract

338.85 g of 95% ethanol soluble extract of pine tree leaf prepared in Step 1-1, was suspended in 1.8 L of distilled water and 1.8 L of hexane was added thereto to fractionate into hexane layer and residue, three times. In a similar fractionation methods except for using 1.8 L of methylene chloride, ethyl acetate, and n-butanol, fractionation was performed to afford a methylene chloride layer, ethyl acetate layer, and n-butanol layer, respectively. The fractionated hexane layer, methylene chloride layer, ethyl acetate layer, n-butanol layer, and water layer were collected and concentrated in vacuo to obtain three non-polar solvent soluble fractions, i.e., (a) 95.18 g of hexane soluble fraction (designated as “S11-H”, hereinafter), (b) 46.11 g of methylene chloride soluble fraction (designated as “S11-M”, hereinafter), (c) 19.42 g of ethyl acetate soluble fraction (designated as “S11-E”, hereinafter); and polar-solvent soluble fractions, i.e., (d) 57.94 g of n-butanol soluble fraction (designated as “S11-B”, hereinafter) and (e) 90.2 g of water soluble fraction (designated as “S11-W”, hereinafter).

1-3. Preparation of Purified Extract

30 g of 95% ethanol soluble extract of pine tree leaf prepared in Step 1-1, was suspended in 30 ml of distilled water and performed to HP-20 column chromatography (Diaion). At first, 2 L of distilled water used as a mobile phase was added to the column to afford the 1st purified extract eluted by water and subsequently, 2 L of 30% ethanol, 2 L of 50% ethanol, 2 L of 70% ethanol, 2 L of 95% ethanol, 2 L of acetone and 2 L of methylene chloride, were eluted to afford the 2nd purified extract eluted by 30% ethanol, the 3rd purified extract eluted by 50% ethanol, the 4th purified extract eluted by 70% ethanol, the 5th purified extract eluted by 95% ethanol, the 6th purified extract eluted by acetone and the 7th purified extract eluted by methylene chloride, respectively.

The 6th purified extract eluted by acetone and the 7th purified extract eluted by methylene chloride were mixed together and the other purified extracts were concentrated under vacuo to obtain 7.96 g of the 1st purified extract eluted by water (designated as “S11-HPO”, hereinafter), 3.44 g of the 2nd purified extract eluted by 30% ethanol (designated as “S11-HP30”, hereinafter), 2.97 g of the 3rd purified extract eluted by 50% ethanol (designated as “S11-HP50”, hereinafter), 1.75 g of the 4th purified extract eluted by 70% ethanol (designated as “S11-HP70”, hereinafter), 6.35 g of the 5th purified extract eluted by 95% ethanol (designated as “S11-HP95”, hereinafter), and 6 g of the 6th purified extract eluted by acetone and methylene chloride (designated as “S11-HPAM”, hereinafter), respectively.

### Example 2

### Preparation of the Compounds Isolated from Pine Tree Leaf Extract

2-1. Preparation of Compound (a)

46.11 g of methylene chloride soluble extract of pine tree leaf prepared in Example 1, was performed to silica gel column chromatography [column size (75 cm×9 cm); stationary phase (230-400 mesh silica gel); eluting solvent=(1) hexane:ethyl acetate=7:1˜1:1→(2) hexane:ethyl acetate:MeOH=10:10:0.5→(3) methylene chloride:MeOH=1:1] to obtain 594.4 mg of purified 8th and 9th fractions. The fractions were further performed to silica gel column chromatography using by eluting solvent (hexane:ethyl acetate=10:1) and 182.8 mg of 8th fraction was further purified by using LiChroprep RP-18 column chromatography (40-63 μm, Merck, U.S.A., eluting solution: 60% ethanol) to afford 48 mg of novel compound, i.e., 9,14-dihydroxytotara-7-ene-8-oic acid (designated as “compound (a)”, hereinafter) showing following physico-chemical data.

<Compound A>
9,14-dihydroxytotara-7-ene-8-oic acid
   colorless amorphous powder;
   ESIMS m/z (positive): 359 [M+Na]⁺
   ESIMS m/z (negative): 335 [M-H]⁺

¹H-NMR (CDCl₃, 500 MHz) δ 0.97 (3H, d, J=7.0 Hz, H-17), 0.97 (3H, d, J=7.0 Hz, H-16), 1.13 (3H, s, H-20), 1.32 (3H, s, H-19), 1.39 (1H, m, H-13a), 1.43 (1H, m, H-11a), 1.57 (3H, m, H-2, 12a), 1.67 (5H, m, H-1, 3, 11b), 1.88 (1H, septet, J=7.0 Hz, H-15), 2.02 (1H, m, H-13b), 2.06 (1H, dd, J=12.3, 4.8 Hz. H-5), 2.29 (1H, ddd, J=13.0, 9.5, 4.5 Hz, H-12b), 2.41 (1H, m, H-6a), 2.78 (1H, m, H-6b), 6.13 (1H, br s, H-7);

¹³C-NMR (CDCl₃, 125 MHz) δ 17.3 (q, C-17), 17.6 (q, C-16), 17.7 (q, C-20), 17.8 (t, C-2), 18.0 (q, C-19), 20.8 (t, C-11), 23.7 (t, C-12), 25.4 (t, C-6), 27.6 (t, C-13), 32.4 (d, C-15), 33.7 (t, C-1), 37.6 (t, C-3), 38.7 (s, C-10), 40.3 (d, C-5), 47.3 (s, C-4), 79.9 (s, C-14), 82.3 (s, C-9), 127.2 (d, C-7), 144.0 (s, C-8), 184.7 (s, C-18).

2-2. Preparation of Compound (b)

46.11 g of methylene chloride soluble extract of pine tree leaf prepared in Example 1, was performed to silica gel column chromatography [column size (75 cm×9 cm); stationary phase (230-400 mesh silica gel); eluting solvent=(1) hexane:ethyl acetate=7:1˜1:1→(2) hexane:ethyl acetate:MeOH=10:10:0.5→(3) methylene chloride:MeOH=1:1] to obtain 954.3 mg of purified 11th fraction. The fraction were further performed to silica gel column chromatography using by eluting solvent (hexane:ethyl acetate=10:1→1:1), and 145.6 mg of 6th fraction was further purified by using LiChroprep RP-18 column chromatography (40-63 μm, Merck, U.S.A., eluting solution: 50% acetonitrile) to afford 16.3 mg of 9th sub-fraction, and the sub-fraction was further purified by using LiChroprep RP-18 column chromatography (40-63 μm, Merck, U.S.A., eluting solution: 50% acetonitrile) to afford 12.9 mg of novel compound, i.e., (13S)-15-hydroxylabd-8(17)-en-18-oic acid (designated as “compound (b)”, hereinafter) showing following physico-chemical data.

<Compound B>
(13S)-15-hydroxylabd-8(17)-en-18-oic acid
   pale yellow amorphous oil
   ESIMS m/z (positive): 322 [M+Na]⁺
   ESIMS m/z (negative): 298 [M-H]⁺

¹H-NMR (CDCl₃,500 MHz) δ 0.70 (3H, s, H-20), 0.89 (3H, br d, J=6.4 Hz, H-16), 1.14 (3H, m, H-19), 1.93 (1H, dd, H-5), 2.05 (1H, td, H-7a), 2.33 (2H, br d, H-7), 3.66 (2H, m, H-15), 4.50 (1H, br s, H-17a), 4.81 (1H, br s, H-17b)

¹³C-NMR (CDCl₃,125 MHz) δ 14.9 (C-20), 16.5 (C-19), 18.6 (C-2), 20.0 (C-16), 20.9 (C-11), 27.0 (C-6), 30.5 (C-13), 36.4 (C-12), 37.3 (C-3), 38.1 (C-1), 38.2 (C-7), 39.2 (C-10), 39.7 (C-14), 47.7 (C-4), 49.7 (C-5), 57.5 (C-9), 61.4 (C-15), 107.1 (C-17), 148.3 (C-8), 185.0 (C-18)

2-3. Preparation of Compounds (c)-(g)

46.11 g of methylene chloride soluble extract of pine tree leaf prepared in Example 1, was performed to silica gel column chromatography [column size (75 cm×9 cm); stationary phase (230-400 mesh silica gel); eluting solvent: (1) hexane:ethyl acetate=7:1˜1:1→(2) hexane:ethyl acetate:MeOH=10:10:0.5→(3) methylene chloride:MeOH=1:1] to obtain 594.4 mg of purified 8th and 9th fractions (Fr. A); 494.4 mg of purified 10th fraction (Fr. B); 954.3 mg of purified 11th fraction (Fr. C); 761.3 mg of purified 13th fraction (Fr. D) and 1.92 g of 15th and 16th fraction (Fr. E).

The purified fraction (Fr. A) were further performed to silica gel column chromatography (hexane:ethyl acetate=10:1, 5:1, 3:1, and 1:1) and LiChroprep RP-18 column chromatography (40-63 μm, Merck, U.S.A., eluting solution: 60% acetonitrile) to afford 61.9 mg of the 1st sub-fractions. The sub-fraction was further purified by using LiChroprep RP-18 column chromatography (40-63 μm, Merck, U.S.A., eluting solution: 60% methanol) to afford 5.6 mg of ent-labd-8(17)-ene-15,18-dioic acid (designated as “compound (c)”, hereinafter).

494.4 mg of the 10th fraction (Fr.B) was further purified using silica gel column chromatography (hexane:ethyl acetate=10:1) to afford 380.7 mg of 6th sub-fraction. The purified 6th sub-fraction was further performed to LiChroprep RP-18 column chromatography (40-63 μm, Merck, U.S.A., eluting solution: 70% methanol) to afford 93.7 mg of the 2nd and 3rd sub-fractions, and the subfractions were further purified by LiChroprep RP-18 column chromatography (40-63 μm, Merck, U.S.A., eluting solution: 60% acetonitrile) to afford 15.4 mg of 13-oxo-15,16-dinorlabda-8(17),11E-dien-19-oic acid (designated as “compound (d)”, hereinafter).

954.3 mg of purified 11th fraction (Fr. C) was further purified using silica gel column chromatography (hexane:ethyl acetate=10:1˜1:2) to afford 145.6 mg of 6th sub-fraction. The purified 6th sub-fraction were further performed to LiChroprep RP-18 column chromatography (40-63 μm, Merck, U.S.A., eluting solution: 50% acetonitrile) to afford 13.3 mg of the 2nd sub-fraction, and the subfraction were further purified by Sephadex LH-20 column chromatography (eluting solvent: methanol) to afford 7.9 mg of 13-hydroxy-8,11,13-podocarpatrien-18-oic acid (designated as “compound (e)”, hereinafter). 761.3 mg of purified 13rd fraction (Fr. D) was further purified using LiChroprep RP-18 column chromatography (40-63 μm, Merck, U.S.A., eluting solution: 70% acetonitrile) to afford 368.8 mg of the 12th and 13rd sub-fractions, and the sub-fractions were further purified by LiChroprep RP-18 column chromatography (40-63 μm, Merck, U.S.A., eluting solution: 60% acetonitrile) to afford 20.2 mg of 13-hydroxy-8,11,13-podocarpatrien-18-oic acid (designated as “compound (f)”, hereinafter).

1.92 g of purified 15th and 16th fraction (Fr. E) was further purified silica gel column chromatography (hexane:ethyl acetate=10:1˜1:1) to afford 257.6 mg of 5th sub-fraction, and the sub-fraction was purified by using LiChroprep RP-18 column chromatography (40-63 μm, Merck, U.S.A., eluting solution: 50% acetonitrile) to afford 53.6 mg of 7-oxo-15-hydroxydehydroabietic acid (designated as “compound (g)”, hereinafter), of which chemical structures were identified comparing with the physico-chemical data published in the literatures.

<Compound (c)>
ent-labd-8(17)-ene-15,18-dioic acid [Ref.: Wen-Chiung Su, et al., Phytochemistry, 41 (1), 1996, 255-261]
[α]_{D}+23.3 (CHCl₃, c0.15)
ESI MS (positive) m/z 359 [M+Na]+

¹H-NMR (CDCl₃, 500 MH) δ 0.70 (H-20), 0.97 (H-16), 1.15 (H-15), 4.50, 4.82 (H-17)

¹³C-NMR (CDCl₃, 125 MH) δ 14.9 (C-20), 16.5 (C-2), 18.6 (C-16), 20.1 (C-11), 20.9 (C-6), 27.0 (C-18), 30.9 (C-13), 35.7 (C-12), 37.3 (C-3), 38.0 (C-1), 38.2 (C-7), 39.2 (C-10), 41.3 (C-14), 47.7 (C-4), 49.7 (C-5), 57.2 (C-9), 107.2 (C-17), 148.1 (C-8), 178.9 (C-15), 184.7 (C-19).

<Compound (d)>
13-oxo-15,16-dinorlabda-8(17),11E-dien-19-oic acid [Ref.: Takahiro Katoh, et al., Chem. Pharm. Bull, 50(12), 2002, 1625-1629]
[α]_{D}+22.52 (CHCl₃, c0.1)
ESI MS (positive) m/z 313 [M+Na]+

¹H-NMR (CDCl₃, 500 MH) δ 0.92 (H-20), 1.20 (H-19), 2.28 (H-14), 4.44, 4.81 (H-17), 6.10 (H-12), 6.84 (H-11)

¹³C-NMR (CDCl₃, 125 MH) δ 15.5 (C-19), 16.8 (C-6), 18.2 (C-2), 25.6 (C-20), 27.6 (C-14), 36.3 (C-7), 37.3 (C-3), 38.8 (C-10), 39.8 (C-1), 47.5 (C-4), 48.7 (C-5), 60.8 (C-9), 109.5 (C-17), 134.0 (C-12), 145.7 (C-11), 147.9 (C-8), 184.5 (C-18), 198.2 (C-13)

<Compound (e)>
13-hydroxy-8,11,13-podocarpatrien-18-oic acid [Ref.: H. T. Andrew Cheung, et al., Tetrahedron, 49(36), 1993, 7903-7915]
[α]_{D}+12.4 (CHCl₃, c 0.9)
ESI MS (positive) m/z 294 [M+Na]+

¹H-NMR (CDCl₃, 500 MH) δ 1.19 (H-20), 1.28 (H-19), 6.48 (H-14), 6.60 (H-12), 7.09 (H-11)

¹³C-NMR (CDCl₃, 125 MH) δ 16.4 (C-19), 18.7 (C-2), 21.8 (C-6), 25.4 (C-20), 30.1 (C-7), 36.8 (C-10), 36.9 (C-1), 38.3 (C-5), 44.9 (C-3), 47.5 (C-4), 113.2 (C-12), 115.1 (C-14), 125.7 (C-11), 136.8 (C-8), 142.2 (C-9), 153.2 (C-13), 184.5 (C-18)

<Compound (f)>
7α-hydroxycallitirisic acid [Ref.: Ching-Kuo Lee, et al., Pytochemistry, 4, 1994, 983-986]
m.p. 111.5-113.5
[α]_{D}+47.6 (CHCl₃, c0.1)
ESI MS (positive) m/z 340 [M+Na]+

¹H-NMR (CDCl₃, 500 MH) δ 1.22 (H-16, H-17), 1.23 (H-20), 1.27 (H-19), 2.28 (H-7), 7.10 (H-12) 7.14 (H-11), 7.38 (H-14)

¹³C-NMR (CDCl₃, 125 MH) δ 16.5 (C-19), 18.6 (C-6), 24.1 (C-16), 24.2 (C-17), 25.6 (C-20), 32.9 (C-2), 33.9 (C-15), 36.5 (C-1), 37.7 (C-7), 38.1 (C-10), 43.6 (C-5), 47.4 (C-4), 70.8 (C-3), 124.3 (C-11), 125.4 (C-14), 126.0 (C-12), 137.6 (C-8), 146.7 (C-13), 146.8 (C-9), 184.0 (C-18)

<Compound (g)>
7-oxo-15-hydroxydehydroabietic acid [Ref.: William A. ayer and John B. macaulay, Canadian Journal of Chemistry, 65(7), 1987, 7-14]
[α]_{D}+16.79 (CHCl₃, c0.3)
ESI MS (positive) m/z 323 [M+Na]+

¹H-NMR (CDCl₃, 500 MH) δ 1.25 (H-20), 1.33 (H-18), 1.56 (H-16, H-17), 7.33 (H-12), 7.70 (H-11), 8.04 (H-14)

¹³C-NMR (CDCl₃, 125 MH) δ 16.4 (C-18), 18.3 (C-2), 23.8 (C-20), 31.7 (C-16), 31.7 (C-17), 36.7 (C-5), 37.2 (C-1), 37.5 (C-3), 37.9 (C-10), 43.7 (C-6), 46.5 (C-4), 72.5 (C-15), 123.4 (C-12), 123.8 (C-14), 130.6 (C-11), 130.9 (C-8), 147.5 (C-13), 154.0 (C-9), 182.8 (C-19), 198.9 (C-7)

2-4. Preparation of Compounds (h)-(l)

95.18 g of hexane soluble extract of pine tree leaf prepared in Example 1, was performed to silica gel column chromatography [column size (75 cm×9 cm); stationary phase (230-400 mesh silica gel); eluting solvent: (1) hexane: methylene chloride=1:1→(2) methylene chloride→(3) hexane:methylene chloride:MeOH=(10:10:0.5→10:10:2)→(4) methylene chloride: MeOH=1:1] to obtain 4.06 g of purified 5th and 6th fractions. The purified 5th and 6th fractions were further purified by using silica gel column chromatography [eluting solvent: (1) hexane:ethyl acetate=(20:1˜3:1)→(2) methylene chloride:MeOH=3:1→(3) MeOH] to afford 58.8 mg of ent-18-hydrorxy-13-epimanoyl oxide (designated as “compound (h)”, hereinafter).

46.11 g of methylene chloride soluble extract of pine tree leaf prepared in Example 1, was performed to silica gel column chromatography [column size (75 cm×9 cm); stationary phase (230-400 mesh silica gel); eluting solvent=(1) hexane:ethyl acetate=7:1˜1:1→(2) hexane:ethyl acetate:MeOH=10:10:0.5→(3) methylene chloride:MeOH=1:1] to obtain 405.9 mg of purified 6th and 7th fractions (Fr. A) and 864.7 mg of purified 12th fraction (Fr. B). The purified fractions (Fr. A) were further performed to silica gel column chromatography using by eluting solvent (hexane:ethyl acetate=10:1) to afford 74.9 mg of the 11th, 12th and 13rd sub-fractions. The sub-fractions were further purified by using LiChroprep RP-18 column chromatography (40-63 μm, Merck, U.S.A., eluting solution: 80% MeOH) to afford (1) 4 mg of dehydroabietic acid (designated as “compound (i)”, hereinafter), (2) 21.1 mg of sandaracopimaric acid (designated as “compound (j)”, hereinafter) and (3) 2.9 mg of 15-hydroxydehydroabietic acid (designated as “compound (k)”, hereinafter)

864.7 mg of the 12th fraction (Fr. B) was further purified using by Sephadex LH-20 chromatography (eluting solution: methylene chloride:MeOH=4:6) to afford 441 mg of sub-fraction, and the sub-fraction was further purified by using LiChroprep RP-18 column chromatography (40-63 μm, Merck, U.S.A., eluting solution: 80% MeOH) to afford 106.3 mg of caryophyllene oxide (designated as “compound (l)”, hereinafter), of which chemical structures were identified comparing with the physico-chemical data published in the literatures.

<Compound (h)>
ent-18-hydrorxy-13-epimanoyl oxide [Ref: Donald, B. S. et al., Phytochemistry, 27 (2), 1988, 517-522; Antonio, G. G. et al., Phytochemistry, 16, 1977, 107-110]
m.p. 47-48° C.;
[α]_{D}+13.1 (CHCl₃, c0.013);
ESI MS (positive) m/z 329 [M+Na]+;

¹H-NMR (CDCl₃, 500 MH) δ 0.72 (3H, s, H-20), 0.97 (3H, s, H-16), 1.13 (3H, s, H-17), 1.20 (3H, s, H-19), 3.44 (1H, d, J=10.5H, H-18a), 3.67 (aH, d, J=10.5H, H-18b), 4.91 (1H, br d, J=11.0H, H-15a), 4.96 (1H, br d, J=17.5H, H-15b), 6.0 (1H, dd, J=17.5, 11.0H, H-14);

¹³C-NMR (CDCl₃, 125 MH) δ 16.2 (C-11), 16.5 (C-20), 18.5 (C-2), 20.4 (C-6), 24.0 (C-17), 27.1 (C-18), 32.9 (C-16), 35.0 (C-12), 36.0 (C-4), 37.0 (C-10), 38.9 (C-3), 39.7 (C-1), 43.7 (C-7), 57.3 (C-5), 58.8 (C-9), 65.6 (C-19), 73.5 (C-13), 76.1 (C-8), 109.8 (C-15), 147.9 (C-14).

<Compound (i)>
dehydroabietic acid [Ref: Miguel, A. G. et al., Eur. J. Med. Chem., 45, 2010, 811-816]
[α]_{D}+59.7 (CHCl₃, c0.5);
ESI MS (positive) m/z 323 [M+Na]+;

¹H-NMR (CDCl₃, 500 MH) δ 1.21 (3H, s, H-20), 1.22 (6H, d J=6.0H, H-16, 17), 1.27 (3H, s, H-19), 2.82 (1H, m, H-15), 6.88 (1H, br s, H-14), 6.88 (1H, d, J=1.5H, H-14), 7.0 (1H, dd, J=8.0, 4.0H, H-12), 7.17 (1H, d, J=8.0H, H-11);

¹³C-NMR (CDCl₃, 125 MH) δ 16.5 (C-19), 18.8 (C-2), 20.0 (C-6), 24.2 (C-16,17), 25.3 (C-20), 30.2 (C-7), 33.7 (C-15), 37.0 (C-3), 37.1 (C-10), 38.2 (C-1), 44.8 (C-5), 47.7 (C-4), 124.1 (C-12), 124.3 (C-11), 127.1 (C-14), 134.9 (C-8), 146.0 (C-13), 147.0 (C-9), 185.3 (C-18).

<Compound (j)>
sandaracopimaric acid [Ref.: Norio, M. et al., Biosci. Biotechnol. Biochem., 72(2), 2008, 477-484]
m.p. 162-163° C.;
[α]_{D}-5.2 (CHCl₃, c0.43);
ESI MS (positive) m/z 324 [M+Na]+;

¹H-NMR (CDCl₃, 500 MH) δ 0.84 (3H, s, H-20), 1.04 (3H, s, H-17), 1.21 (3H, s, H-19), 4.89 (1H, dd, J=11.0, 1.5H, H-16b) 4.91 (1H, dd, J=17.5, 1.5H, H-16a), 5.22 (1H, s, H-14), 5.77 (1H, dd, J=17.5, 11.0H, H-15);

¹³C-NMR (CDCl₃, 125 MH) δ 15.4 (C-20), 17.0 (C-19), 18.4 (C-2), 18.8 (C-11), 25.1 (C-6), 26.2 (C-17), 34.7 (C-12), 35.7 (C-7), 37.3 (C-3), 37.6 (C-13), 38.0 (C-10), 38.5 (C-1), 47.5 (C-4), 49.0 (C-5), 50.8 (C-9), 110.4 (C-16), 129.4 (C-14), 136.8 (C-8), 149.1 (C-15), 185.5 (C-18).

<Compound (k)>
15-hydroxydehydroabietic acid [Ref.: Lai-King, Sy. et al., J. Nat. Prod., 61, 1998, 907-912]
[α]_{D}+113.7 (CHCl₃, c0.02);
ESI MS (positive) m/z 339 [M+Na]+;

¹H-NMR (CDCl₃, 500 MH) δ 1.20 (3H, s, H-20), 1.26 (3H, br s, H-19), 1.54 (6H, s, H-16,17), 7.13 (1H, br s, H-14), 7.20 (2H, m, H-11,12);

¹³C-NMR (CDCl₃, 125 MH) δ 16.5 (C-19), 18.7 (C-2), 21.9 (C-5), 25.3 (C-20), 30.4 (C-7), 31.8 (C-10), 37.1 (C-16,17), 38.1 (C-3), 44.8 (C-1), 47.6 (C-6), 51.0 (C-4), 72.6 (C-15), 122.2 (C-14), 124.4 (C-12), 125.1 (C-11), 135.0 (C-8), 146.2 (C-13), 148.1 (C-9), 184.8 (C-18).

<Compound (l): Caryophyllene Oxide>
caryophyllene oxide [Ref.: Thebtaranonth, C. Y. et al., Phytochemistry, 40(1), 1995, 125-128]
m.p. 60-62° C.;
[α]_{D} −70.12 (CHCl₃, c0.65);
ESI MS (positive) m/z 243 [M+Na]+;

¹H-NMR (CDCl₃, 500 MH) δ 0.99 (3H, s, H-12), 1.02 (3H, s, H-13), 1.21 (3H, s, H-14), 4.87 (1H, m, H-15a), 4.99 (1H, m, H-1513);

¹³C-NMR (CDCl₃, 125 MH) δ 21.7 (C-13), 27.2 (C-2), 29.8 (C-7), 30.0 (C-12), 30.3 (C-6), 34.0 (C-11), 39.2 (C-3), 39.8 (C-10), 48.8 (C-9), 50.7 (C-1), 63.7 (C-5), 112.8 (C-15), 151.8 (C-8).

### Reference Example 1

### Preparation of Reagents

1-1. Cell Preparation

293TT cell to use for HPV pseudovirus reproduction and in vitro assay (Schiller Lab.)—i.e., a manipulated 293T cell line prepared by transforming human embryonic kidney cell by adenovirus E1a and expressing the cell by SV40 large T antigen, was incubated with Dulbeccos modified Eagles medium (DMEM; SH30243, Hyclone, UT, USA) supplemented with heat inactivated 10% FBS (26140079, Hyclone, UT, USA) and maintained at 37° C. under the condition of providing 5% CO₂ gas.

1-2. Production of HPV-16 pseudovirus

1-2-1. Plasmid

: for In vitro antiviral assay, HPV-SEAP pseudovirus was produced and for In vivo challenge test, HPV-Luc PV was produced in the test. To produce HPV-SEAP PV, p-SEAP and p16L1L2 plasmid, and HPV-Luc PV, pc-Luc and p16L1L2 plasmid were used. Each plasmid was procured from Schiller Lab (Laboratory of Cellular Oncology, Center for Cancer Research, and National Cancer Institute, Bethesda (USA).

1-2-2. Transfection

:5×10⁶ 293TT cell was seeded on 75T flask and incubated at 37° C., for 16 hours in 5% CO₂ atmosphere. 19 μg of p16L1/L2 and 19 μg of pSEAP or pc-Luc plasmid were cotransfected by Lipofectin Reagent (18292-011, Invitrogen, CA, USA). 6 hours after the transfection, the cell was changed with complete media, cultivated at 37° C., for 48 hours, and harvested by trypsinization. The harvested cell was washed with Dulbecco's Phosphate-Buffered Saline (DPBS, 14190-250, Invitrogen, CA, USA).

1-2-3. Cell Harvest and Virion Maturation

: The harvested cell was resuspended in DPBS 1 ml, and 5% Triton X-100 (9002-93-1, Sigma, Mo., USA), 25 mM ammonium sulphate (pH 9, A4418, Sigma-Aldrich, MO, USA) and 0.2% benzonase (9025-65-4, Sigma, UK) were added thereto. The cell was incubated for 24 hours at 37° C. to mature the virus.

1-2-4. Salt Extraction

: The maturated virion was cooled with ice for 5 mins and 0.17 volume of 5N NaCl was added thereto to incubate for 20 mins again. The virion solution was collected, transferred to e-tube, centrifuged at 4° C. with the speed of 12,000 rpm for 10 mins and collect the supernatant to subject to opti-prep ultracentrifugation or keep at −80° C.

1-2-5. Purification

: 77 ml of SIGMA density gradient medium was mixed with 23 ml of DPBS/0.8M NaCl to make 46% optiprep gradient and 37%, 33%, 39% gradient solution were made to DPBS/0.8M NaCl solution in a similar way, (27%:9.3 ml DPBS/0.8M NaCl+13.2 ml 46% Optiprep, 33%:6.4 ml DPBS/0.8M NaCl+16.1 ml 46%, 39%:3.4 ml DPBS/0.8M NaCl+19 ml 46%). 1 ml of each 39%, 33%, and 27% gradient solution were added to 5 ml of Beckman ultracentrifuge tube (361625, Beckman Coulter, USA) carefully in order not to braking the layers, and 1 ml of the virion supernatant was loaded thereon. The solution was centrifuged with the speed of 47,800 rpm, at 16° C., for 4 hours using by Ultracentrifuge (Optima L 90K, Beckman Coulter Ultracentrifuge, USA). The virion fraction was collected and kept at −80° C.

1-2-6. HPV PVs Titration

: 5×10³ of 293TT cell was seeded on 96-well plate and incubated at 37° C., for 16 hours in 5% CO₂ atmosphere. After HPV PVs was performed to 5-fold serial dilution, each cell was infected by the virus and incubated for 72 hours. To determine the titer of HPV-SEAP PV, the supernatant of the cell culture was used for determine the activity of secreted alkaline phosphatase (SEAP) using by Great EscAPE™ SEAP Chemiluminescence Kit (631738, Clontech, CA, USA). To determine the titer of HPV-Luc PV, the supernatant of the cell culture was used for determine the luciferase activity using by BioLuxGaussia Luciferase Assay Kit (0301008, New England biolabs, MA, USA). For the chemiluminescent detection, RLU (relative light units) value was obtained using by Luminescence coulter (Micro beta triLux 1450, PerkinElmer, CT, USA) to calculate each HPV PVs titers.

1-3. In Vitro Antiviral Assay

: For the screening assay of samples prepared in Examples and HPV inhibition assay, all the test were performed according to the methods disclosed in the literatures (Shaneyfelt et al., Virology Journal, 3:68, 2006, 1-11; Roden et al., J. Virol., 70, 1996, 5875-5883; Unckell et al., J. Virol., 71, 1997, 2934-2939; Touze et al., Nucleic Acids Research, 26, 1998, 1317-1323; Selinka et al., J. Virol., 77, 2003, 12961-12967; Klasse et al., Journal of General Virology, 83, 2002, 2091-2108)

Prior to in vitro antiviral assay, 5×10³ of 293TT cell was seeded on 96-well plate and incubated at 37° C. for 16 hours in 5% CO₂ atmosphere. The culture media was removed and each extract was diluted to the concentration of 100 μg/ml or 50 μg/ml to distribute into each cell at the dose of 100 μl/cell. After treating with the extract for 16 hours, the culture media was removed and the cell was washed with PBS twice. The cell was infected by 106RLU/ml of HPV PVs at the dose of 100 μl/cell and incubated for 48 hours at 37° C. in 5% CO₂ atmosphere. To determine the viral activity, the supernatant of the cell culture was used for determine the activity of secreted alkaline phosphatase (SEAP) using by Great EscAPE™ SEAP Chemiluminescence Kit (631738, Clontech, CA, USA). To determine the SEAP activity, relative light units (RLU) value was obtained using by Luminescence coulter (Micro beta triLux 1450, PerkinElmer, CT, USA) and the RLU values of the cell treated with the extract and untreated with the extract, were compared with each other. The decrease of SEAP activity was regarded as the inhibition effect against HPV PVs.

1-4. Administration

: 6-weeks old female Balb/C mouse (Orientbio Co., Korea) was used in the experiment and the test samples were orally and topically administrated as follows:

For oral administration, the testing extract mixed with 0.5% methyl cellulose (Sigma, Mo., USA) was orally administrated for 5 days, once a day at the dose of 300 mg/kg. 4 hours after the final administration, HPV 16 PVs was injected into the subcutaneous region of the abdomen. For topical administration, the testing extract mixed with 0.5% methyl cellulose, was topically administrated into the genital tract, for 3 days once a day, at the dose of 150 mg/kg. 4 hours after the final administration, HPV16 PVs was injected into the genital tract. The mouse untreated with testing extract was used as a negative control.

1-5. Psudovirus Challenge

: The challenge test was performed using by HPV PVs and the luciferase gene ex-pressing HPV16-Luc PVs which is prepared by several steps, i.e., production, maturation, extraction, purification, and titration (http://home.ccr.cancer.gov/lco/). 4 days before the challenge, 3 mg of Depot medroxyprogesterone acetate (Depo-Provera™) (Pharmacia, Belgium) was injected into mice, and 3% carboxymethylcellulose (Sigma Aldrich, MO, USA) and 4% Nonoxinol-9 (Sigma Aldrich, MO, USA) were used when the HPV16-Luc PVs challenged (Roberts et al., Nature medicine, 13, 2007, 857-861). To orally administrated mice with test sample, HPV16-Luc PVs was subcutaneously injected to the abdomen at the dose of 5×10⁶ RLU. To topically administrated mice with test samples, 6 hours before the HPV16-Luc PVs challenge, 20 μl of 4% nonoxynol-9 was intravaginally administrated into the mice and the HPV16-Luc PVs mixed with 20 μl of 3% carboxymethylcellulose was injected to the vaginal tract of mouse at the dose of 5×10⁶ RLU. 3 days after the challenge, all the mice was anesthetized and 30 μl of luciferin (caliper, MA, USA, 7 mg/ml) was intraperitoneally injected into the orally administrated mice. HPV16-Luc PVs allows the ability of pseudoinfection when the luciferase gene transferring plasmid, pLucf, is encapsidated (Roberts et al., Nature medicine, 13, 2007, 857-861). After being left for 10 mins, the luciferase expression of each mouse was detected with IVIS 200 bioluminescence imaging system (Xenogen, NJ, USA) and the image was compared with each other. The expression of luciferase of the image was quantitatively determined with Living Image 2.20 software (Xenogen, NJ, USA) and the preventive efficacy of each test sample against HPV was compared with each other.

### Experimental Example 1

### Virus Inhibition Test (In Vitro)

To determine the inhibitory effect on luciferase-comprising HPV virus contagion of the test samples prepared in Examples, following test was performed according to the method disclosed in the literature (Shaneyfelt et al., Virology Journal, 3:68, 2006, 1-11; Roden et al., J. Virol., 70, 1996, 5875-5883; Unckell et al., J. Virol., 71, 1997, 2934-2939; Touze et al., Nucleic Acids Research, 26, 1998, 1317-1323; Selinka et al., J. Virol., 77, 2003, 12961-12967; Klasse et al., Journal of General Virology, 83, 2002, 2091-2108)

5×10³293TT cell was seeded on 96-well plate and incubate for 16 hours. 50 μg of test sample was mixed with the culture media in the concentration of 100 ug/ml and incubated for 16 hours. The media was washed with 100 μl of PBS twice and the cell was infected with 10⁶RLU/ml of HPV pseudovirion at the dose of 100 μl/cell to incubate at 37° C., for 48 hours in 5% CO₂ atmosphere.

5× lysis buffer in Great EscAPE™ SEAP Chemiluminescence Kit (631738, Clontech, CA, USA) was made to 1× and both of 45 μl of 1× lysis buffer and 15 μl of cell culture medium were added to 96-well plate (3912, Costar, NY, USA). 60 μl of the substrate in Great EscAPE™ SEAP Chemiluminescence kit was added thereto and the relative light units (RLU) value was obtained using by Luminescence coulter (Micro beta triLux 1450, PerkinElmer, CT, USA). The RLU values of the cell treated with the extract and untreated with the extract, were compared with each other and the decrease of SEAP activity was regarded as the inhibition effect against HPV PVs.

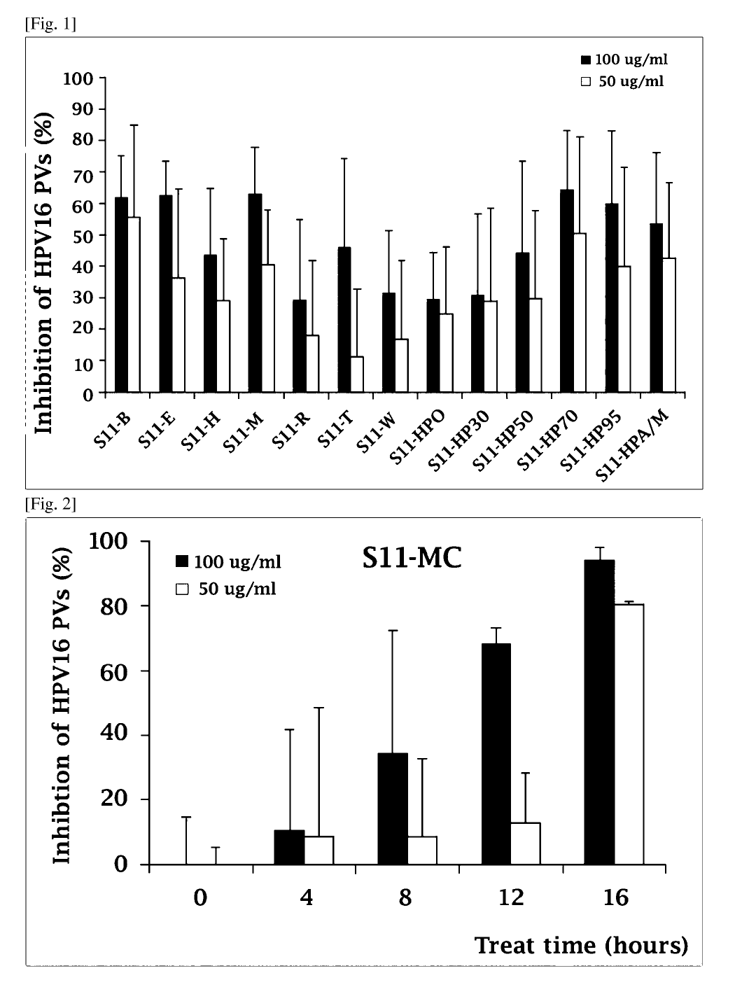

At the result, 100 μg/ml of S11-B, S11-E, S11-M, S11-HP70, and S11-HP95 showed more than 50% inhibitory activity from the virus, in particular, 50 μg/ml of S11-B and S11-HP70 showed more than 50% inhibitory activity from HPV16 PVs. (See FIG. 1)

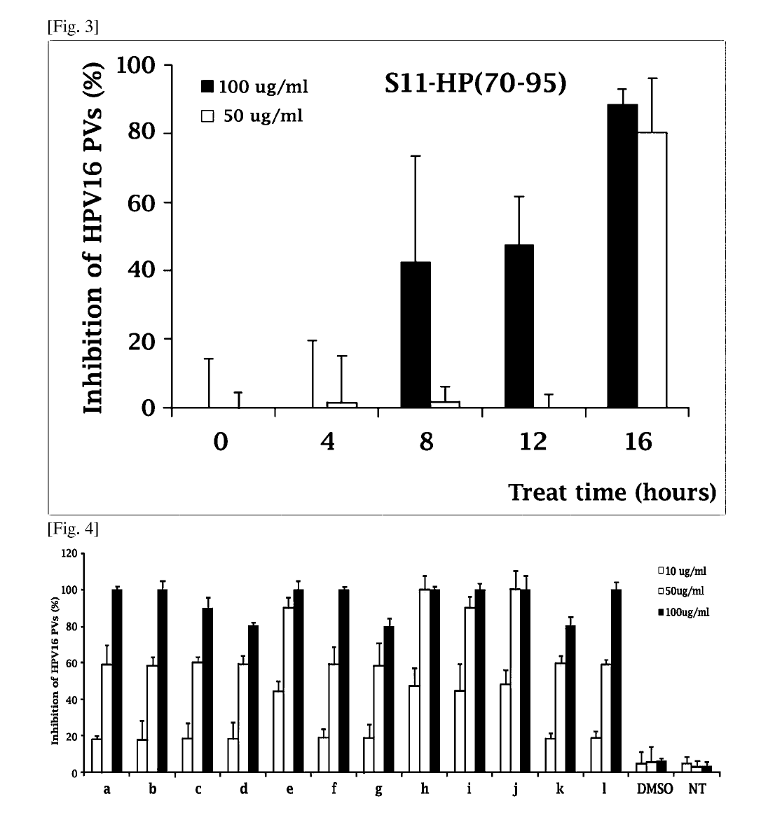

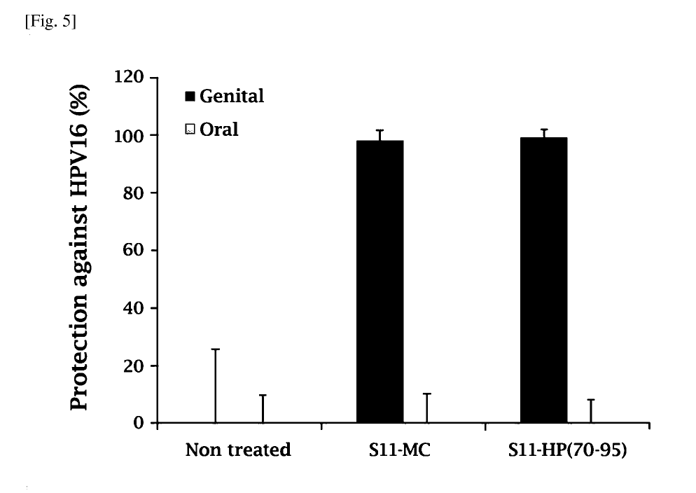

All the compounds isolated from pine tree extract, i.e., 9,14-dihydroxytotara-7-ene-8-oic acid (a), (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l), showed potent inhibitory effect on HPV16 PVs ranging from 70 to 80% inhibitory activity at 50 μg/ml, in particular, dehydroabietic acid (h), sandaracopimaric acid (i) showed about 50% inhibitory activity at 5 μg/ml. (See FIG. 4).

### Experimental Example 2

### Virus Inhibition Test According to Pre-Treatment Time

To determine the inhibitory effect on luciferase-comprising HPV virus contagion of the test samples prepared in Examples according to pre-treatment time, following test was performed according to the method disclosed Experimental Example 1.

Both of two test samples, (1) S11-MC and (2) S11-HP(70-95) were diluted to 100 μg/ml and 50 μg/ml, and 293TT cell was pre-treated with the test samples, for 0, 4, 8, 12, and 16 hours. The culture medium in each well was removed, washed with PBS twice and infected by 10⁶ RLU/ml of HPV 16 PVs. 48 hours after the infection, the SEAP activity of each cell culture was determined to evaluate the inhibition of HPV 16 PVs.

As can be seen in FIG. 2,3 showing the inhibition ratio of test samples expressed by percentage (%) against HPV16 PVs, 50 μg/ml of S11-MC inhibited by 8% in case of treating for 4 to 8 hours, 12% for 12 hours, and 80% for 16 hours; 100 μg/ml of S11-MC inhibited by 10% in case of treating for 4 hours, 34% for 8 hours, 68% for 12 hours, and 94% for 16 hours. 100 μg/ml of S11-HP(70-95) inhibited by 42% in case of treating for 8 hours, 47% for 12 hours, and 88% for 16 hours (See FIG. 2,3).

### Experimental Example 3

### Cytotoxicity Test Against Human Tumor Cell Line

To determine the cytotoxicity against human tumor cell line of the test samples prepared in Examples, following sulforhodamin B (SRB) bioassy test was performed according to the method disclosed in the literature (Skehan, P., et al., J. Natl. Cancer Inst., 82, 1990, 1107-1112).

Various human tumor cell lines, i.e., A-549 (human adenocarcinoma of lung, NCI), SK-OV-3 (human ovarian tumor, NCI), SK-MEL-2 (human malignant melanoma, NCI), HCT15 (human colon adenocarcinoma, NCI), MES-SA (human uterine carcinoma, NCI) and MES-SA/DX5 (multidrug resistant carcinoma subline of MES-SA, NCI) were used in the test.

The subcultured cell lines were detached from the surface of wall using by trypsin-EDTA solution and adjusted to various concentration of cell lines, i.e., 5×10³ cells/well (A-549, HCT15), 1×10⁴ cells/well (SK-MEL-2), 5×10³ cells/well (SK-OV-3) in 96-well flat bottom microplate. The inoculated cell lines were incubated in CO₂ incubator (MCO-20AIC, SANYO Electric Co., LTd.) to be attached on the bottom of incubator and the culture media was removed with aspirator. 6 logarithmic concentrations of the diluted test samples were added to each well containing cell line at 100 μl/well in a three fold dilution manner and the cell was incubated for 48 hours. The culture media in each well was removed and 100μ of 10% trichloroacetic acid was added thereto to be left alone at 4° C. for 1 hour and to fix the cells to the bottom of the plates. After fixing the cell, the plate was washed with distilled water five to six times to remove remaining trichloroacetic acid solution and dry completely. the staining solution dissolving 0.4% SRB (sulforhodamine B, Sigma) solution in 1% acetic acid solution at the dose of 100 μl/well, was added to the completely dried plates to stain for 30 mins and the plates were washwd with 1% acetic acid 5 to six times to remove excess SRB which did not bind to the cell. the stained cell plate was dried at room temperature and 10 mM trisma base solution was added to each well at the dose of 100 μl/well. The cell plates were shaked with titer plate shaker (KOMA Orbital Shaker KE011, KOMABIOTech) for 10 mins to exude the staining solution and to determine the absorbance at 520 nm using by microplate spectrophotometer (Sunrise, TECAN).

At the result, it has been confirmed that the test samples prepared in Examples showed potent cytotoxicity against various human tumor cell line, i.e., MES-SA (human uterine carcinoma), and MES-SA/DX5 (multidrug resistant carcinoma subline of MES-SA) (See Table 1 and 2).

As can bee seen in Table 3 and 4, 9,14-dihydroxytotara-7-ene-8-oic acid (a), as well as (13S)-15-hydroxylabd-8(17)-en-18-oic acid (b), ent-labd-8(17)-ene-15,18-dioic acid (c), 13-oxo-15,16-dinorlabda-8(17),11E-dien-19-oic acid (d), 13-hydroxy-8,11,13-podocarpatrien-18-oic acid (e), 7α-hydroxycallitirisic acid (f), 7-oxo-15-hydroxydehydroabietic acid (g), ent-18-hydrorxy-13-epimanoyl oxide (h), dehydroabietic acid (i), sandaracopimaric acid (j), 15-hydroxydehydroabietic acid (k), and caryophyllene oxide (l), showed potent cytotoxic activity on all the tumor cell lines, i.e., A-549 (human adenocarcinoma of lung, NCI), SK-OV-3 (human ovarian tumor, NCI), SK-MEL-2 (human malignant melanoma, NCI), HCT15 (human colon adenocarcinoma, NCI), MES-SA (human uterine carcinoma, NCI) and MES-SA/DX5 (multidrug resistant carcinoma subline of MES-SA, NCI).

TABLE 1
Cytotoxicity data of the extract and solvent fractions of pine
tree leaf against MES-SA(human uterine carcinoma), and MES-
SA/DX5 (multidrug resistant carcinoma subline of MES-SA)

| | IC₅₀(μg/ml) | | | | | | |
|---|---|---|---|---|---|---|---|
| Cell line | S11-T | S11-H | S11-MC | S11-E | S11-B | S11-W | doxorubicin |
| MES-SA | 22.69 | 11.04 | 11.73 | 112.47 | 257.39 | >300.0 | 0.0002 |
| MES-SA/DX5 | 23.14 | 16.28 | 12.92 | 186.94 | >300.0 | >300.0 | 0.4122 |
| Ratio* | 1.02 | 1.47 | 1.10 | 1.66 | — | — | 2061 |

*Ratio is the value calculated by dividing the value of MES-SA/DX5 value by that of MES-SA.

TABLE 2
Cytotoxicity data of the purified extracts of pine tree
leaf against on MES-SA(human uterine carcinoma), and MES-
SA/DX5 (multidrug resistant carcinoma subline of MES-SA)

| | IC₅₀(μg/ml) | | | | | | |
|---|---|---|---|---|---|---|---|
| Cell line | HP0 | HP30 | HP50 | HP70 | HP95 | HPAM | doxorubicin |
| MES-SA | >300.0 | >300.0 | 188.63 | 56.67 | 13.44 | 15.83 | 0.0002 |
| MES-SA/DX5 | >300.0 | >300.0 | 286.32 | 81.35 | 11.94 | 16.70 | 0.4122 |
| Ratio* | — | — | 1.52 | 1.44 | 0.89 | 1.05 | 2061 |

*Ratio is the value calculated by dividing the value of MES-SA/DX5 value by that of MES-SA.

TABLE 3
Cytotoxicity data of the novel compound (a) and known compounds (h)~(l)
isolated from pine tree leaf against various cancer cell lines.

| | IC₅₀(μg/ml) | | | | | |
|---|---|---|---|---|---|---|
| test sample | A-549 | SK-OV-3 | SK-MEL-2 | HCT15 | MES-SA | MES-SA/DX5 |
| compound (a) | 127.16 | 120.05 | 99.45 | 117.31 | 77.22 | 80.57 |
| compound (h) | 13.34 | 12.70 | 11.26 | 10.04 | 8.17 | 9.54 |
| compound (i) | 13.44 | 17.29 | 13.02 | 15.68 | 10.33 | 12.01 |
| compound (j) | 13.64 | 12.14 | 11.17 | 13.58 | 10.45 | 11.07 |
| compound (k) | 109.07 | 141.77 | 88.82 | 98.25 | 90.66 | 91.92 |
| compound (l) | 36.36 | 23.71 | 18.64 | 26.75 | 15.25 | 17.73 |
| Doxorubicin | 0.0072 | 0.0104 | 0.0012 | 0.0833 | 0.0051 | 1.45 |

TABLE 4
Cytotoxicity data of the novel compound (b) and known compounds (c)~(g)
isolated from pine tree leaf against various cancer cell lines

| | IC₅₀(μg/ml) | | | | | |
|---|---|---|---|---|---|---|
| test sample | MES-SA | MES-SA/DX5 | Ratio* | HCT15 | HCT15/CL02 | Ratio** |
| compound (b) | 19.07 | 11.52 | 0.60 | 20.16 | 12.85 | 0.64 |
| compound (c) | >30.0 | >30.0 | — | >30.0 | 21.47 | <0.72 |
| compound (d) | >30.0 | >30.0 | — | >30.0 | 27.64 | <0.92 |
| compound (e) | >30.0 | 25.84 | <0.86 | >30.0 | 26.37 | <0.88 |
| compound (f) | 26.81 | 27.44 | 1.02 | >30.0 | >30.0 | — |
| compound (g) | >30.0 | >30.0 | — | >30.0 | >30.0 | — |
| Doxorubicin | 0.0051 | 1.45 | 284.31 | 0.016 | 2.11 | 131.88 |

*Ratio was the value calculated by dividing the value of MES-SA/DX5 by that of MES-SA.
**Ratio was the value calculated by dividing the value of HCT15/CL02 by that of HCT15.

### Experimental Example 4

### Virus Inhibition Test (In Vivo)

To determine the inhibitory effect on the mouse treated with luciferase-comprising HPV of the test samples prepared in Examples, following test was performed according to the method disclosed in the literature (Roberts, J. N., et al., (2007). Genital transmission of HPV in a mouse model is potentiated by nonoxynol-9 and inhibited by carrageenan. Nat Med 13; 857-861.).

4-1. Administration

: 6-weeks old female Balb/C mouse (Orientbio Co., Korea) was used in the experiment and the test samples were orally and topically administrated as follows:

For oral administration, the testing extract mixed with 0.5% methyl cellulose (Sigma, Mo., USA) was orally administrated for 5 days, once a day at the dose of 300 mg/kg. 4 hours after the final administration, HPV 16 PVs was injected into the subcutaneous region of the abdomen. For topical administration, the testing extract mixed with 0.5% methyl cellulose, was topically administrated into the genital tract, for 3 days once a day, at the dose of 150 mg/kg. 4 hours after the final administration, HPV16 PVs was injected into the genital tract. The mouse untreated with testing extract was used as a negative control.

4 days before the challenge, 3 mg of Depot medroxyprogesterone acetate (Depo-Provera™) (Pharmacia, Belgium) was injected into the back region of mice at the dose of 3 mg/mouse.

4-2. Abdomen Challenge Test

For topical administration, the testing extract mixed with 0.5% methyl cellulose, was topically administrated into the vaginal tract, for 3 days once a day, at the dose of 150 mg/kg. 4 hours after the final administration, the HPV16-Luc PVs mixed with 1% carboxymethylcellulose was subcutaneously injected to the abdomen of mouse. Three days after the infection, the mice were anesthetized and 30 μl of luciferin (caliper, MA, USA) in the concentration of 7 mg/ml was intraperitoneally injected to shoot the image using by IVIS 200 bioluminescence imaging system (Xenogen, NJ, USA) 10 mins after the injection.

For oral administration, the testing extract mixed with 0.5% methyl cellulose (Sigma, Mo., USA) was orally administrated for 5 days, once a day at the dose of 300 mg/kg. 4 hours after the final administration, HPV16 PVs mixed with 1% carboxymethylcellulose (1:1, Sigma Aldrich, MO, USA) was injected into the subcutaneous region of the abdomen. Further imaging procedure was identical to that disclosed in topical ad-ministration.

4-3. Intravaginal Challenge Test

6 hours before the HPV16-Luc PVs challenge, 20 μl of 4% nonoxynol-9 (Sigma Aldrich, MO, USA) was intravaginally administrated into the mice and the HPV16-Luc PVs mixed with 20 μl of 3% carboxymethylcellulose (Sigma Aldrich, MO, USA) was injected to the vaginal tract of mouse at the dose of 5×10⁶ RLU. 3 days after the challenge, all the mice was anesthetized and 30 μl of luciferin (caliper, MA, USA, 7 mg/ml) was intraperitoneally injected into the orally administrated mice. HPV16-Luc PVs allows the ability of pseudoinfection when the luciferase gene transferring plasmid, pLucf, is encapsidated (Roberts et al., Nature medicine, 13, 2007, 857-861). After being left for 10 mins, the luciferase expression of each mouse was detected with IVIS 200 bioluminescence imaging system (Xenogen, NJ, USA) and the image was compared with each other. The expression of luciferase of the image was quantitatively determined with Living Image 2.20 software (Xenogen, NJ, USA) and the preventive efficacy of each test sample against HPV was compared with each other.

4-4. Result

6 weeks-old female mouse which is not treated with test sample was used as a negative control and the HPV16 PVs challenge method was performed by the identical method in the above.

The mouse challenged through vaginal tract showed the regional expression of luciferase on genital area and that subcutaneously challenged into the abdomen showed the expression of luciferase on overall body including injected area. Accordingly, it has been confirmed that the luciferase activity has been detected by HPV 16 PVs contagion and the test sample showed potent preventive effect from the contagion since the negative control showed some luminescence.

The topically administrated mice with S11-MC did not show luminescence and therefore the test sample prevented completely from HPV16 PVs contagion. For oral administration, the testing extract (S11-MC) mixed with 0.5% methyl cellulose (Sigma, Mo., USA) was orally administrated for 5 days, once a day at the dose of 300 mg/kg. 4 hours after the final administration, HPV16 PVs mixed with 1% carboxymethylcellulose (1:1, Sigma Aldrich, MO, USA) was injected into the subcutaneous region of the abdomen. Further imaging procedure was identical to that disclosed in topical administration. The orally administrated mice with S11-MC showed some luminescence and therefore the test sample did not prevent from HPV16 PVs contagion.

The testing extract [S11-HP (70-95)] mixed with 0.5% methyl cellulose was topically administrated into vaginal tract for 3 days, once a day at the dose of 150 mg/kg. The mice topically administrated with S11-HP(70-95) did not show any luminescence and therefore the test sample completely prevent from HPV16 PVs contagion.

It has been confirmed that the amount of luminescence in the test groups topically administrated with S11-MC [symbol] S11-HP(70-95) showed less than 2% comparing with that in negative control group (FIG. 2,3).

### Experimental Example 5

### Acute Toxicity Test of Oral Administration in Rat

The acute toxicity test was performed by administrating inventive extract or compounds to 6-weeks aged SPF Sprague-Dawley rats.

250 mg/kg, 500 mg/kg, 1000 mg/kg, 5000 mg/kg of inventive extract or compounds was orally administrated to each group consisting of 2 rats and the symptoms of rats were observed for 14 days. After administrating the extract or compounds, all the clinical changes i.e., mortality, clinical signs, body weight changes was observed and blood test such as haematological test and hematological biochemistry test was performed. The abnormal changes of abdominal organ and thoracic organ were observed after autopsy.

There did not show any changes in mortality, clinical signs, body weight changes and gross findings in any group or either gender. Furthermore, there showed any toxicity in test group treated with 5000 mg/kg of inventive extract or compounds.

Accordingly, it has been confirmed that the inventive extract or compounds prepared in the present invention was potent and safe substance showing LD₅₀ (more than 5000 mg/kg) in oral administration.

### MODE FOR THE INVENTION

Hereinafter, the formulating methods and kinds of excipients will be described, but the present invention is not limited to them. The representative preparation examples were described as follows.

Preparation of Injection

| | |
|---|---|
| S11-T extract | 100 mg |
| Sodim methabifulfite | 3.0 mg |
| Methyl paraben | 0.5 mg |
| Propyl paraben | 0.1 mg |
| Distilled water for injection | optimum amount |

Injection preparation was prepared by dissolving active component, controlling pH to about 7.5 and then filling all the components in 2 ml ample and sterilizing by conventional injection preparation method.

Preparation of Powder

| | |
|---|---|
| S11-H extract | 500 mg |
| Corn Starch | 100 mg |
| Lactose | 100 mg |
| Talc | 10 mg |

Powder preparation was prepared by mixing above components and filling sealed package.

Preparation of Tablet

| | |
|---|---|
| S11-MC extract | 200 mg |
| Corn Starch | 100 mg |
| Lactose | 100 mg |
| Magnesium stearate | optimum amount |

Tablet preparation was prepared by mixing above components and entabletting.

Preparation of Capsule

| | |
|---|---|
| HP95 extract | 100 mg |
| Lactose | 50 mg |
| Corn starch | 50 mg |
| Talc | 2 mg |
| Magnesium stearate | optimum amount |

Tablet preparation was prepared by mixing above components and filling gelatin capsule by conventional gelatin preparation method.

Preparation of Liquid

| | |
|---|---|
| HPAM extract | 1000 mg |
| Sugar | 20 g |
| Polysaccharide | 20 g |
| Lemon flavor | 20 g |

Liquid preparation was prepared by dissolving active component, and then filling all the components in 1000 ml ample and sterilizing by conventional liquid preparation method.

Preparation of Health Care Food

| HP0 | 1000 | mg |
|---|---|---|
| Vitamin mixture | optimum amount | |
| Vitamin A acetate | 70 | g |
| Vitamin E | 1.0 | mg |
| Vitamin B₁ | 0.13 | mg |
| Vitamin B₂ | 0.15 | mg |
| Vitamin B6 | 0.5 | mg |
| Vitamin B12 | 0.2 | g |
| Vitamin C | 10 | mg |
| Biotin | 10 | g |
| Amide nicotinic acid | 1.7 | mg |
| Folic acid | 50 | g |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above mentioned vitamin and mineral mixture may be varied in may ways. Such variations are not to be regarded as a departure from the spirit and scope of the present invention.

Preparation of Health Beverage

| | | |
|---|---|---|
| compound (a) | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the extract of pine leaf extract or the compound isolated therefrom showed potent inhibitory effect on HPV virus as well as anti-cancer effect on various cancer diseases through various in vitro test and in vivo tests, therefore, it can be used as the effective and safe therapeutics or health food for treating and preventing cancer disease.

1. A 9,14-dihydroxytotara-7-ene-8-oic acid of chemical formulae (a) or a pharmacologically acceptable salt thereof:

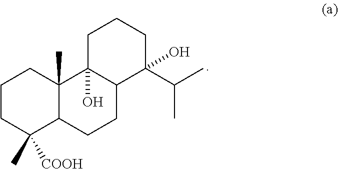

2. A (13S)-15-hydroxylabd-8(17)-en-18-oic acid of chemical formulae (b) or a pharmacologically acceptable salt thereof:

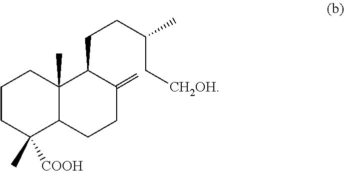 [SMILES: C=C1CCC2[C@](C)(C(=O)O)CCC[C@]2(C)[C@H]1CC[C@H](C)CCO]

3. A composition comprising an extract of pine tree leaf or a compound selected from the group consisting of 9,14-dihydroxytotara-7-ene-8-oic acid, (13S)-15-hydroxylabd-8(17)-en-18-oic acid, ent-labd-8(17)-ene-15,18-dioic acid, 13-oxo-15,16-dinorlabda-8(17), 11E-dien-19-oic acid, 13-hydroxy-8,11,13-podocarpatrien-18-oic acid, 7α-hydroxycallitirisic acid, 7-oxo-15-hydroxydehydroabietic acid, ent-18-hydrorxy-13-epimanoyl oxide, dehydroabietic acid, sandaracopimaric acid, 15-hydroxydehydroabietic acid, and caryophyllene oxide, or a pharmacologically acceptable salt thereof.

4. The composition of claim 3, wherein said extract is selected from the group consisting of a crude extract, polar solvent soluble extract, non-polar solvent soluble extract, and purified extract of pine tree leaf.

5. The composition of claim 4, wherein said crude extract is prepared by extracting plant material with water, lower alcohols such as methanol, ethanol, or a mixture thereof.

6. The composition of claim 4, wherein said non-polar solvent soluble extract is prepared by suspending the crude extract in 0.005 to 10-fold volume (v/w) of distilled water, and fractionating the suspension with a non-polar solvent repeatedly to give the non-polar solvent soluble extract.

7. The composition of claim 4, wherein said polar solvent soluble extract is prepared by fractionating the crude extract with a polar solvent; removing a non-polar solvent soluble fraction; and collecting a polar solvent soluble fraction to give the polar solvent soluble extract.

8. The composition of claim 4, wherein said purified extract is selected from the group consisting of (1) a purified extract eluted with water (designated as “S11-HPO” hereinafter), (2) a purified extract eluted with 30% ethanol (designated as “S11-HP30” hereinafter), (3) a purified extract eluted with 50% ethanol (designated as “S11-HP50” hereinafter), (4) a purified extract eluted with 70% ethanol (designated as “S11-HP70” hereinafter), (5) a purified extract eluted with 95% ethanol (designated as “S11-HP95” hereinafter), or (6) a purified extract eluted with acetone and methylene chloride (designated as “S11-HPAM” hereinafter) using by adsorbent resin and eluting solvent with decreasing the polarity of the solvent starting from water, ethanol, acetone to methylene chloride, serially.

9. The composition of claim 3, wherein said pine tree is selected from Pinus densiflora Sieb. et Zucc, P. rigida, P. taeda, P. thunberii Palatore, P. koraiensis Sieb. et Zucc, Pinus palustris Miller, Pinus palustris Miller, P. pinaster Aiton, P. sylvestris L., P. laricid Poiret, or P. longifolia Rocvurgh.

10. (canceled)

11. (canceled)

12. (canceled)

13. A method of treating or preventing a cancer disease in human or in a mammal in need thereof, the method comprising administering a therapeutically effective amount of the composition of claim 3 as an effective ingredient, together with a pharmaceutically acceptable carrier thereof.

14. The composition of claim 3 which is a health functional food or health care food.

15. The composition of claim 14, wherein said health functional food is in powder, granule, tablet, capsule or beverage type.

16. (canceled)

17. The composition of claim 14, wherein said health care food is provided as food, health beverage, or dietary supplement.

18. A method for preparing purified extract of claim 3, comprising the step of
adding about 1-30 fold weight (w/w) distilled water to the crude extract of pine tree leaf;
performing adsorption purification method using by adsorbent resin selected from the group consisting of SP207, HP20SS, Diaion HP 20, SP-850 resin, active carbon, and Amberlite XAD-2,4; and
eluting the column with an eluting solvent starting from water, ethanol, acetone to methylene chloride, serially, to give (1) a purified extract eluted with water (S11-HPO), (2) a purified extract eluted with 30% ethanol (S11-HP30), (3) a purified extract eluted with 50% ethanol (S11-HP50), (4) a purified extract eluted with 70% ethanol (S11-HP70), (5) a purified extract eluted with 95% ethanol (S11-HP95), and (6) a purified extract eluted with acetone and methylene chloride (S11-HPAM).

19. The composition of claim 3, which is a pharmaceutical composition and comprises a pharmaceutically acceptable carrier.

20. The method of claim 13, wherein the cancer is a cervical cancer disease caused by human papillomavirus.

21. The method of claim 13, wherein said cancer disease is selected from the group consisting of a cervical cancer, resistant cervical cancer, lung cancer, ovarian tumor, malignant melanoma, colonic cancer, colon cancer or rectal cancer, bone cancer, pancreatic cancer, skin cancer, cancer of the head and neck, cutaneous or intraocular melanoma, uterine cancer, ovarian cancer, rectal cancer or cancer of the anal region, stomach cancer, colon cancer, breast cancer, gynecologic tumors, Hodgkin's disease, cancer of the esophagus, cancer of the small intestine, cancer of the endocrine system, sarcomas of soft tissues, cancer of the urethra, cancer of the penis, prostate cancer, chronic or acute leukemia, solid tumors of childhood, lymphocytic lymphonas, cancer of the bladder, cancer of the kidney or ureter, or neoplasms of the central nervous system.
